(19) United States
(12) Patent Application Publication
(10) Pub. No.: US 2020/0016091 A1
(43) Pub. Date: Jan. 16, 2020

(54) WATER-SOLUBLE CANNABIDIOL

(71) Applicant: Orochem Technologies, Inc., Naperville, IL (US)

(72) Inventors: Anantha Krishna MALLIA, Rockford, IL (US); David W. HOUSE, Arlington Heights, IL (US); Anil Rajaram OROSKAR, Oak Brook, IL (US); Asha Anil OROSKAR, Oak Brook, IL (US)

(73) Assignee: Orochem Technologies, Inc., Naperville, IL (US)

(21) Appl. No.: 16/036,271

(22) Filed: Jul. 16, 2018

(51) Int. Cl. A61K 31/05; A61K 47/40; A61K 9/10; A61K 9/00

(52) U.S. Cl. CPC A61K 31/05; A61K 47/40; A61K 47/10; A61K 9/0053; A61K 9/10

(57) ABSTRACT

Provided is a composition for oral consumption comprising (a) methylated-β-cyclodextrin and/or hydroxypropylated-β-cyclodextrin, (b) cannabidiol, and (c) water. Also provided is a method of making a composition for oral consumption comprising (a) methylated-β-cyclodextrin and/or hydroxypropylated-β-cyclodextrin, (b) cannabidiol, and (c) water.

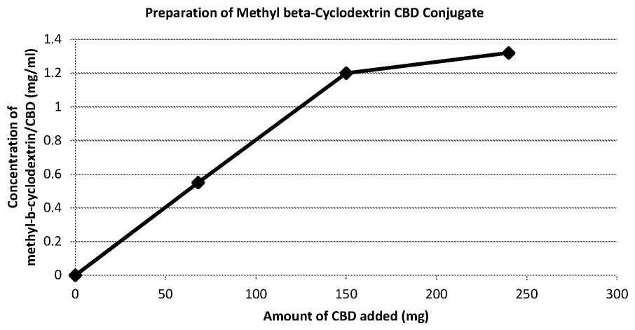

### BACKGROUND

Cannabidiol (“CBD”) is a major constituent obtained from the processing of cannabis or hemp that has anti-convulsive, anti-anxiety, anti-psychotic, anti-nausea, and anti-rheumatoid arthritic properties. CBD can be isolated directly from cannabis or hemp, or, for example, from heating cannabidiolic acid (“CBDA”), which is another major constituent of cannabis or hemp. The terms “hemp” and “cannabis” refer to the genus Cannabis, which contains three species Cannabis sativa, Cannabis indica, and Cannabis ruderalis. All three species are of the family Cannabaceae, which also includes the genus Humulus, or hops.

Contrary to tetrahydrocannabinol (“THC”), which is the principal psychoactive component of cannabis or hemp, CBD does not appear to have psychoactive properties, and thus, CBD is an attractive candidate as a therapeutic agent. One complication with CBD is that it has a strong preference for non-aqueous media, dissolving only in oil and organic solvents, such as alcohol-based solvents, aliphatic hydrocarbons, dimethyl sulfoxide, dimethyl formamide, and acetone. CBD's lack of solubility in water results in low absorption and bioavailability, thereby presenting a challenge for administration as an orally ingested therapeutic.

In order to increase bioavailability and absorption, CBD conventionally has been formulated with a number of excipients (e.g., cyclodextrin, maltodextrin, polyphosphates, glycerin, polysorbate, starch, acacia gum, gum arabic, etc.). However, inclusion of such ingredients can be costly, result in wasted materials, and hinder the manufacturing process.

Therefore, there is a need for a new formulation for oral consumption of CBD that has improved absorption and bioavailability, which is relatively easy to manufacture, and reduces the amount of wasted materials during the preparation process.

It will be appreciated that this background description has been created by the inventors to aid the reader, and is not to be taken as a reference to prior art nor as an indication that any of the indicated problems were themselves appreciated in the art. While the described principles can, in some regards and embodiments, alleviate the problems inherent in other systems, it will be appreciated that the scope of the protected innovation is defined by the attached claims, and not by the ability of the claimed invention to solve any specific problem noted herein.

### BRIEF SUMMARY

The present invention provides cannabidiol with enhanced water-solubility. In various embodiments, the invention provides compositions for oral consumption (e.g., liquid preparations), methods of preparing the oral compositions, and mixtures (a precursor composition in some embodiments) used in methods of preparing the final oral compositions. The final composition includes cannabidiol, cyclodextrin component (which is generally substituted, e.g., methylated or hydroxypropylated), and water. The oral compositions can be non-alcoholic or alcoholic (i.e., additionally containing ethanol, if desired). The present inventors have found a preferred desired proportion of the cannabidiol relative to cyclodextrin component to produce a desirably high concentration of water soluble cannabidiol with a relatively low amount of wasted material (e.g., excess solubilizing agent or additional ingredients). In preferred embodiments, the oral composition is prepared with a relatively low amount of cannabidiol and cyclodextrin as compared with conventional techniques so that the user experiences lower intake of cyclodextrin material or other agent, thereby optimizing cannabidiol intake relative to other components that may be unwanted or undesirable for ingestion.

Thus, in one aspect, the invention provides a composition for oral consumption (e.g., in liquid form) comprising, consisting essentially of, or consisting of (a) methylated-β-cyclodextrin, (b) cannabidiol, and (c) water, wherein the cannabidiol is present in an amount from about 20 mg to about 40 mg per gram of methylated-β-cyclodextrin, and the cannabidiol is present in the composition in an amount from about 0.8 mg/mL to about 1.6 mg/mL.

In another aspect, the invention also provides a composition for oral consumption (e.g., in liquid form) comprising, consisting essentially of, or consisting of (a) hydroxypropylated-β-cyclodextrin, (b) cannabidiol, and (c) water, wherein the cannabidiol is present in an amount from about 0.1 mg to about 3.5 mg per gram of hydroxypropylated-β-cyclodextrin, and the cannabidiol is present in the composition in an amount from about 0.01 mg/mL to about 0.13 mg/mL.

In another aspect, the invention provides a mixture used in a method for making the oral composition. In some embodiments, the mixture comprises (a) methylated-β-cyclodextrin, (b) cannabidiol, (c) ethanol, and (d) water, wherein the mixture comprises cannabidiol in an amount from about 20 mg to about 40 mg per gram of methylated-β-cyclodextrin, and ethanol in an amount from about 5 mL to about 50 mL per 100 mL of water; and such mixture can be used for making a composition for oral consumption comprising: (a) methylated-β-cyclodextrin, (b) cannabidiol, and (c) water, wherein the cannabidiol is present in an amount from about 20 mg to about 40 mg per gram of methylated-β-cyclodextrin, and the cannabidiol is present in the composition in an amount from about 0.8 mg/mL to about 1.6 mg/mL. In some embodiments, the mixture comprises (a) methylated-β-cyclodextrin, (b) cannabidiol, (c) ethanol, and (d) water, wherein the mixture comprises cannabidiol in an amount from about 25 mg to about 30 mg per gram of methylated-β-cyclodextrin, and ethanol in an amount from about 5 mL to about 50 mL per 100 mL of water; and such mixture can be used for making a composition for oral consumption comprising: (a) methylated-β-cyclodextrin, (b) cannabidiol, and (c) water, wherein the cannabidiol is present in an amount from about 25 mg to about 30 mg per gram of methylated-β-cyclodextrin, and the cannabidiol is present in the composition in an amount from about 0.8 mg/mL to about 1.6 mg/mL.

In some embodiments, the mixture comprises (a) hydroxypropylated-β-cyclodextrin, (b) cannabidiol, (c) ethanol, and (d) water, wherein the mixture comprises cannabidiol in an amount from about 0.1 mg to about 3.5 mg per gram of hydroxypropylated-β-cyclodextrin, and ethanol in an amount from about 5 mL to about 50 mL per 100 mL of water; and such mixture can be used for making a composition for oral consumption comprising: (a) hydroxypropylated-β-cyclodextrin, (b) cannabidiol, and (c) water, wherein the cannabidiol is present in an amount from about 0.1 mg to about 3.5 mg per gram of hydroxypropylated-β-cyclodextrin, and the cannabidiol is present in the composition in an amount from about 0.01 mg/mL to about 0.13 mg/mL. In some embodiments, the mixture comprises (a) hydroxypropylated-β-cyclodextrin, (b) cannabidiol, (c) ethanol, and (d) water, wherein the mixture comprises cannabidiol in an from about 0.1 mg to about 1.5 mg per gram of hydroxypropylated-β-cyclodextrin, and ethanol in an amount from about 5 mL to about 50 mL per 100 mL of water; and such mixture can be used for making a composition for oral consumption comprising: (a) hydroxypropylated-β-cyclodextrin, (b) cannabidiol, and (c) water, wherein the cannabidiol is present in an amount from about 0.1 mg to about 1.5 mg per gram of hydroxypropylated-β-cyclodextrin, and the cannabidiol is present in the composition in an amount from about 0.01 mg/mL to about 0.13 mg/mL.

In another aspect, the invention further provides a method of making a composition for oral consumption comprising (i) adding methylated-β-cyclodextrin and/or hydroxypropylated-β-cyclodextrin to water to form a solution of methylated-β-cyclodextrin and/or hydroxypropylated-β-cyclodextrin in water; (ii) adding cannabidiol to ethanol to form a solution of cannabidiol in ethanol; (iii) adding the solution of cannabidiol in ethanol to the solution of methylated-β-cyclodextrin and/or hydroxypropylated-β-cyclodextrin in water over a period of at least about 15 minutes to form a mixture; (iv) stirring the mixture for a period of about 6 hours to about 24 hours; (v) filtering the mixture; and (vi) removing the ethanol from the mixture to form the composition for oral consumption comprising (a) methylated-β-cyclodextrin and/or hydroxypropylated-β-cyclodextrin, (b) cannabidiol, and (c) water.

### BRIEF DESCRIPTION OF THE DRAWING

The FIGURE is a graph of the concentration of methylated-β-cyclodextrin/cannabidiol inclusion complex (Y-axis) versus the amount of cannabidiol added (X-axis), as described in Example 1.

### DETAILED DESCRIPTION

Embodiments of the invention provide for a composition for oral consumption (e.g., as a liquid preparation) containing cannabidiol (CBD) and a method of making an oral composition containing the CBD. Advantageously, compositions for oral consumption in accordance with preferred embodiments of the invention exhibit improved absorption and bioavailability. Furthermore, in accordance with preferred embodiments of the invention, the CBD-containing compositions can be prepared efficiently, while reducing the amount of wasted materials and excess CBD required to produce CBD in solution. Thus, embodiments of the invention represent improvements to conventional techniques. In this respect, as described herein, CBD is insoluble in water and therefore requires additional components to facilitate solubilization (“solubilizing agents”). In order to achieve desirable levels of CBD in an aqueous solution, conventional methods have required the addition of excess CBD and excess solubilizing agents. A major drawback with this approach is that excess materials are either consumed unnecessarily, or disposed of unnecessarily. In addition, the length of conventional manufacturing processes is unnecessarily extended due to an excess of materials, thereby creating longer solubilization times and complicated purification protocols.

Contrary to conventional approaches for the preparation of water soluble CBD, preferred embodiments of the process only require four ingredients (i.e., (a) methylated-β-cyclodextrin or hydroxypropylated-β-cyclodextrin, (b) cannabidiol, (c) ethanol, and (d) water). Preferred embodiments of the resulting composition in a simple form for oral consumption only require three ingredients (i.e., (a) methylated-β-cyclodextrin or hydroxypropylated-β-cyclodextrin, (b) cannabidiol, and (c) water although other ingredients can be included if desired). In addition, the process has been optimized such that the ratios of these ingredients produce a relatively high concentration of water soluble CBD with a relatively low (e.g., reduced relative to conventional techniques) amount of wasted materials (e.g., methylated-β-cyclodextrin or hydroxypropylated-β-cyclodextrin and cannabidiol). Other benefits of the inventive process and compositions will be readily apparent from the disclosure provided herein.

Embodiments of the invention provide a composition for oral consumption comprising, consisting essentially of, or consisting of (a) methylated-β-cyclodextrin, (b) cannabidiol, and (c) water, wherein the cannabidiol is present in an amount from about 20 mg to about 40 mg per gram of methylated-β-cyclodextrin, and the cannabidiol is present in the composition in an amount from about 0.8 mg/mL to about 1.6 mg/mL.

In some embodiments, the composition for oral consumption comprises water. The water can be any type of water suitable for oral consumption. For example, the water can be purified (e.g., by filtration, distillation, or reverse osmosis), carbonated, dyed, flavored, or any combination thereof.

The cyclodextrin component can be substituted. In some embodiments, the composition for oral consumption comprises methylated-β-cyclodextrin. As used herein, the terms “methyl-β-cyclodextrin” and “methylated-β-cyclodextrin” can be used interchangeably to refer to a chemical compound of formula:

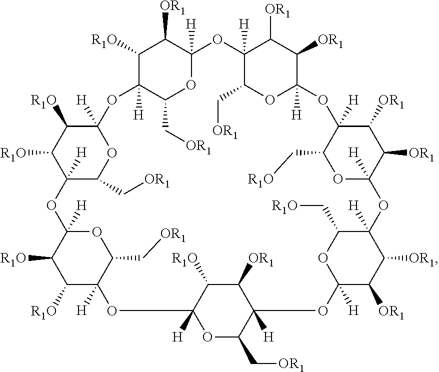

wherein each R₁ independently is hydrogen or methyl. Accordingly, the terms “methyl-β-cyclodextrin” and “methylated-β-cyclodextrin” can refer to any β-cyclodextrin molecule that has from 1 to 21 (e.g., 1, 2, 3, 4, 5, 6, 7, 8, 9, 10, 11, 12, 13, 14, 15, 16, 17, 18, 19, 20, or 21) hydroxyl substituents (i.e., when R₁ is hydrogen) replaced with methoxy substituents (i.e., when R₁ is methyl). Typically, the methylated-β-cyclodextrin is a mixture of compounds with a varying number of hydroxyl substituents replaced with methoxy substituents such that the average number of hydroxyl substituents replaced with methoxy substituents ranges from about 1 to about 21 (e.g., about 1, about 2, about 3, about 4, about 5, about 6, about 7, about 8, about 9, about 10, about 11, about 12, about 13, about 14, about 15, about 16, about 17, about 18, about 19, about 20, or about 21).

Accordingly, in various embodiments, the methylated-β-cyclodextrin can have a weight average molecular weight of, for example, from about 1134 g/mol to about 1429 g/mol (e.g., from about 1134 g/mol to about 1400 g/mol, from about 1134 g/mol to about 1350 g/mol, from about 1134 g/mol to about 1300 g/mol, from about 1134 g/mol to about 1250 g/mol, from about 1134 g/mol to about 1200 g/mol, from about 1134 g/mol to about 1150 g/mol, from about 1150 g/mol to about 1429 g/mol, from about 1200 g/mol to about 1429 g/mol, from about 1250 g/mol to about 1429 g/mol, from about 1300 g/mol to about 1429 g/mol, from about 1350 g/mol to about 1429 g/mol, from about 1400 g/mol to about 1429 g/mol, from about 1200 g/mol to about 1400 g/mol, or from about 1250 g/mol to about 1350 g/mol).

In embodiments where the composition for oral consumption comprises (a) methylated-β-cyclodextrin, (b) cannabidiol, and (c) water, the cannabidiol is present in an amount from about 20 mg to about 40 mg per gram of methylated-β-cyclodextrin. For example, the cannabidiol can be present in an amount from about 22 mg to about 38 mg per gram of methylated-β-cyclodextrin, e.g., from about 23 mg to about 37 mg, from about 24 mg to about 36 mg, from about 25 mg to about 35 mg, from about 25 mg to about 34 mg, from about 25 mg to about 33 mg, from about 25 mg to about 32 mg, from about 25 mg to about 31 mg, from about 25 mg to about 30 mg, from about 26 mg to about 30 mg, from about 27 mg to about 30 mg, or from about 28 mg to about 30 mg per gram of methylated-β-cyclodextrin.

In embodiments where the composition for oral consumption comprises (a) methylated-β-cyclodextrin, (b) cannabidiol, and (c) water, the cannabidiol is preferably present in the composition in an amount from about 0.8 mg/mL to about 1.6 mg/mL. For example, the cannabidiol can be present in an amount from about 0.9 mg/mL to about 1.5 mg/mL based on the volume of the composition, e.g., from about 1 mg/mL to about 1.4 mg/mL, from about 1.1 mg/mL to about 1.3 mg/mL, from about 0.8 mg/mL to about 1.2 mg/mL, or from about 1.2 mg/mL to about 1.6 mg/mL based on the volume of the composition. In preferred embodiments, the cannabidiol is present in the composition in an amount of about 1.2 mg/mL based on the volume of the composition.

Without wishing to be bound by any particular theory, the methylated-β-cyclodextrin forms an inclusion complex with the cannabidiol to make the cannabidiol more soluble in water (e.g., the composition). The amount of cannabidiol, methylated-β-cyclodextrin, and the complex of cannabidiol and methylated-β-cyclodextrin can be analyzed by high-performance liquid chromatography (“HPLC”). A skilled artisan will know suitable parameters for analyzing the composition by HPLC, such that the amounts of cannabidiol and methylated-β-cyclodextrin in the composition can be determined.

Without wishing to be bound by any particular theory, it is believed that in order to achieve a relatively high amount of cannabidiol in solution (i.e., as a complex with methylated-β-cyclodextrin), while wasting a relatively low amount of methylated-β-cyclodextrin and/or cannabidiol, the cannabidiol is present in an amount from about 20 mg to about 40 mg (e.g., about 30 mg) per gram of methylated-β-cyclodextrin and the cannabidiol is present in the composition in an amount from about 0.8 mg/mL to about 1.6 mg/mL (e.g., about 1.2 mg/mL) based on the volume of the composition.

Without wishing to be bound by any particular theory, it is believed that cannabidiol present in the composition comprising methylated-β-cyclodextrin, in a preferred amount from about 0.8 mg/mL to about 1.6 mg/mL (e.g., about 1.2 mg/mL) based on the volume of the composition results in a favorable dosage to be delivered as a composition for oral consumption. Similarly, cannabidiol present in a preferred amount from about 20 mg to about 40 mg (e.g., about 30 mg) per gram of methylated-β-cyclodextrin results in a relatively low amount of unnecessarily disposed starting material (i.e., methylated-β-cyclodextrin and cannabidiol), and also provides a relatively low amount of unnecessarily consumed methylated-β-cyclodextrin.

Typically, in accordance with preferred embodiments, the composition is formed from a mixture comprising: (a) methylated-β-cyclodextrin, (b) cannabidiol, (c) ethanol, and (d) water, wherein the mixture comprises ethanol in an amount from about 5 mL to about 50 mL per 100 mL of water. For example, the mixture can comprise ethanol in an amount from about 5 mL to about 40 mL per 100 mL of water, from about 5 mL to about 30 mL per 100 mL of water, from about 5 mL to about 20 mL per 100 mL of water, from about 10 mL to about 50 mL per 100 mL of water, from about 10 mL to about 40 mL per 100 mL of water, from about 10 mL to about 30 mL per 100 mL of water, or from about 10 mL to about 20 mL per 100 mL of water.

As used herein, the phrase “formed from” refers to the process of converting the mixture, described herein, to the composition, described herein, by any suitable process (e.g., by the removal of ethanol).

In addition, in some embodiments, the mixture comprises cannabidiol in an amount from about 20 mg to about 40 mg per gram of methylated-β-cyclodextrin. For example, the cannabidiol can be present in an amount from about 22 mg to about 38 mg per gram of methylated-β-cyclodextrin, e.g., from about 23 mg to about 37 mg, from about 24 mg to about 36 mg, from about 25 mg to about 35 mg, from about 25 mg to about 34 mg, from about 25 mg to about 33 mg, from about 25 mg to about 32 mg, from about 25 mg to about 31 mg, from about 25 mg to about 30 mg, from about 26 mg to about 30 mg, from about 27 mg to about 30 mg, or from about 28 mg to about 30 mg per gram of methylated-β-cyclodextrin. Without wishing to be bound by any particular theory, it is believed that the cannabidiol and methylated-β-cyclodextrin are added in a favorable amount such that there is not any excess cannabidiol and methylated-β-cyclodextrin to be removed. Accordingly, the composition and the mixture can have the same, or similar, ratios of cannabidiol to methylated-β-cyclodextrin.

In another aspect, the invention also provides a composition for oral consumption comprising, consisting essentially of, or consisting of (a) hydroxypropylated-β-cyclodextrin, (b) cannabidiol, and (c) water, wherein the cannabidiol is present in an amount from about 0.1 mg to about 3.5 mg per gram of hydroxypropylated-β-cyclodextrin, and the cannabidiol is present in the composition in an amount from about 0.01 mg/mL to about 0.13 mg/mL.

Thus, in some embodiments, the substituted cyclodextrin in the composition for oral consumption is in the form of hydroxypropylated-β-cyclodextrin. As used herein, the terms “hydroxypropyl-β-cyclodextrin” and “hydroxypropylated-β-cyclodextrin” can be used interchangeably to refer to a chemical compound of formula:

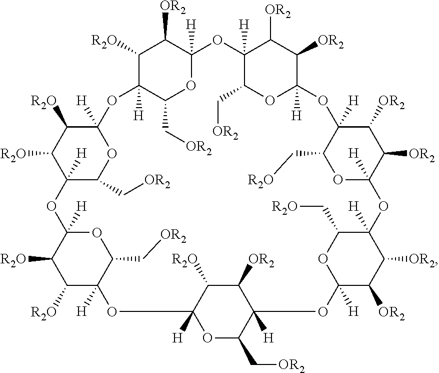

wherein each R₂ independently is hydrogen or hydroxypropyl. Accordingly, the terms “hydroxypropyl-β-cyclodextrin” and “hydroxypropylated-β-cyclodextrin” can refer to any β-cyclodextrin molecule that has from 1 to 21 (e.g., 1, 2, 3, 4, 5, 6, 7, 8, 9, 10, 11, 12, 13, 14, 15, 16, 17, 18, 19, 20, or 21) hydroxyl substituents (i.e., when R₂ is hydrogen) replaced with hydroxypropyl (i.e., when R₂ is hydroxypropyl) substituents. The hydroxypropyl substituent can by any suitable hydroxypropyl substituent (i.e., 1-hydroxypropyl, 2-hydroxypropyl, or 3-hydroxypropyl). In preferred embodiments, the hydroxypropyl substituent is 2-hydroxypropyl. Typically, the hydroxypropylated-β-cyclodextrin is a mixture of compounds with a varying number of hydroxyl substituents replaced with hydroxypropyl substituents such that the average number of hydroxyl substituents replaced with hydroxypropyl substituents ranges from about 1 to about 21 (e.g., about 1, about 2, about 3, about 4, about 5, about 6, about 7, about 8, about 9, about 10, about 11, about 12, about 13, about 14, about 15, about 16, about 17, about 18, about 19, about 20, or about 21).

Accordingly, the hydroxypropylated-β-cyclodextrin can have a weight average molecular weight of, for example, from about 1134 g/mol to about 2354 g/mol (e.g., from about 1134 g/mol to about 2300 g/mol, from about 1134 g/mol to about 2100 g/mol, from about 1134 g/mol to about 1900 g/mol, from about 1134 g/mol to about 1700 g/mol, from about 1134 g/mol to about 1500 g/mol, from about 1134 g/mol to about 1300 g/mol, from about 1300 g/mol to about 2354 g/mol, from about 1500 g/mol to about 2354 g/mol, from about 1700 g/mol to about 2354 g/mol, from about 1900 g/mol to about 2354 g/mol, from about 2100 g/mol to about 2354 g/mol, from about 1400 g/mol to about 2000 g/mol, from about 1500 g/mol to about 1900 g/mol, or from about 1600 g/mol to about 1800 g/mol).

In embodiments where the composition for oral consumption comprises (a) hydroxypropylated-β-cyclodextrin, (b) cannabidiol, and (c) water, the cannabidiol is present in an amount from about 0.1 mg to about 3.5 mg per gram of hydroxypropylated-β-cyclodextrin. For example, the cannabidiol can be present in an amount from about 0.1 mg to about 3 mg per gram of hydroxypropylated-β-cyclodextrin, e.g., from about 0.1 mg to about 2.5 mg, from about 0.1 mg to about 2 mg, from about 0.1 mg to about 1.5 mg, from about 0.1 mg to about 1 mg, from about 0.5 mg to about 3.5 mg, from about 1 mg to about 3.5 mg, from about 0.5 mg to about 3 mg, or from about 1 mg to about 3 mg per gram of hydroxypropylated-β-cyclodextrin.

In embodiments where the composition for oral consumption comprises (a) hydroxypropylated-β-cyclodextrin, (b) cannabidiol, and (c) water, the cannabidiol is present in the composition in an amount from about 0.01 mg/mL to about 0.13 mg/mL based on the volume of the composition. For example, the cannabidiol can be present in an amount from about 0.05 mg/mL to about 0.13 mg/mL, e.g., from about 0.1 mg/mL to about 0.13 mg/mL, from about 0.11 mg/mL to about 0.13 mg/mL, or from about 0.12 mg/mL to about 0.13 mg/mL based on the volume of the composition. In preferred embodiments, the cannabidiol is present in the composition in an amount of about 0.13 mg/mL based on the volume of the composition.

Without wishing to be bound by any particular theory, the hydroxypropylated-β-cyclodextrin forms an inclusion complex with the cannabidiol to make the cannabidiol more soluble in water (e.g., the composition). The amount of cannabidiol, hydroxypropylated-β-cyclodextrin, and the complex of cannabidiol and hydroxypropylated-β-cyclodextrin can be analyzed by high-performance liquid chromatography (“HPLC”). A skilled artisan will know suitable parameters for analyzing the composition by HPLC, such that the amounts of cannabidiol and hydroxypropylated-β-cyclodextrin in the composition can be determined.

Without wishing to be bound by any particular theory, it is believed that in order to achieve a high amount of cannabidiol in solution (i.e., as a complex with hydroxypropylated-β-cyclodextrin), while wasting a relatively low amount of hydroxypropylated-β-cyclodextrin and/or cannabidiol, the cannabidiol is present in an amount from about 0.1 mg to about 3.5 mg (e.g., about 3 mg) per gram of hydroxypropylated-β-cyclodextrin and the cannabidiol is present in the composition in an amount from about 0.01 mg/mL to about 0.13 mg/mL (e.g., about 0.13 mg/mL) based on the volume of the composition.

Without wishing to be bound by any particular theory, it is believed that cannabidiol present in the composition comprising hydroxypropylated-β-cyclodextrin, in an amount from about 0.01 mg/mL to about 0.13 mg/mL (e.g., about 0.13 mg/mL) based on the volume of the composition results in a favorable dosage to be delivered as a composition for oral consumption. Similarly, cannabidiol present in an amount from about 0.01 mg to about 3.5 mg (e.g., about 1.5 mg) per gram of hydroxypropylated-β-cyclodextrin results in a relatively low amount of unnecessarily disposed starting material (i.e., hydroxypropylated-β-cyclodextrin and cannabidiol), and also provides a relatively low amount of unnecessarily consumed hydroxypropylated-β-cyclodextrin.

Typically, the composition is formed from a mixture comprising: (a) hydroxypropylated-β-cyclodextrin, (b) cannabidiol, (c) ethanol, and (d) water, wherein the mixture comprises ethanol in an amount from about 5 mL to about 50 mL per 100 mL of water. For example, the mixture can comprise ethanol in an amount from about 5 mL to about 40 mL per 100 mL of water, from about 5 mL to about 30 mL per 100 mL of water, from about 5 mL to about 20 mL per 100 mL of water, from about 10 mL to about 50 mL per 100 mL of water, from about 10 mL to about 40 mL per 100 mL of water, from about 10 mL to about 30 mL per 100 mL of water, or from about 10 mL to about 20 mL per 100 mL of water.

In addition, the mixture can comprise cannabidiol in an amount from about 0.1 mg to about 3.5 mg per gram of hydroxypropylated-β-cyclodextrin. For example, the cannabidiol can be present in an amount from about 0.1 mg to about 3 mg per gram of hydroxypropylated-β-cyclodextrin, e.g., from about 0.1 mg to about 2.5 mg, from about 0.1 mg to about 2 mg, from about 0.1 mg to about 1.5 mg, from about 0.1 mg to about 1 mg, from about 0.5 mg to about 3.5 mg, from about 1 mg to about 3.5 mg, from about 0.5 mg to about 3 mg, or from about 1 mg to about 3 mg per gram of hydroxypropylated-β-cyclodextrin. Without wishing to be bound by any particular theory, it is believed that the cannabidiol and hydroxypropylated-β-cyclodextrin are added in a favorable amount such that any excess cannabidiol and hydroxypropylated-β-cyclodextrin to be removed is reduced or eliminated. Accordingly, the composition and the mixture can have the same, or similar, ratios of cannabidiol to hydroxypropylated-β-cyclodextrin.

In some embodiments, the composition for oral consumption comprises, consists essentially of, or consists of (a) methylated-β-cyclodextrin, (b) hydroxypropylated-β-cyclodextrin, (c) cannabidiol, and (d) water. Methylated-β-cyclodextrin and hydroxypropylated-β-cyclodextrin can be used in combination in amounts described herein to provide a composition for oral consumption with the desired solubility of cannabidiol. The relative amounts of methylated-β-cyclodextrin, hydroxypropylated-β-cyclodextrin, and cannabidiol can be determined by a skilled artisan depending on the desired amount of cannabidiol in the composition for oral consumption.

In some embodiments, the composition for oral consumption further comprises ethanol. Typically, when the composition for oral consumption comprises ethanol, the ethanol is present in a trace amount. Accordingly, ethanol can be present in an amount of about 0.0001 wt. % or more (e.g., about 0.001 wt. % or more, about 0.01 wt. % or more, about 0.1 wt. % or more, or about 1 wt. % or more). Alternatively, or in addition, ethanol can be present in an amount of about 5 wt. % or less (e.g., about 4 wt. % or less, about 3 wt. % or less, about 2 wt. % or less, about 1 wt. % or less, about 0.1 wt. % or less, about 0.01 wt. % or less, or about 0.001 wt. % or less). Any two of the foregoing endpoints can be used to define a close-ended range, or any single endpoint can be used alone to define an open-ended range. In preferred embodiments, the composition for oral consumption is substantially free of ethanol. As used herein, the phrase “substantially free” refers to no detectable amount.

The composition for oral consumption can further comprise one or more pharmaceutically acceptable excipients. Suitable excipients and the amounts to use may be readily determined under the direction of one of ordinary skill in the art based upon experience and consideration of standard procedures and reference works in the field, e.g., sweetening agents, dyes, flavoring agents, and preservatives.

When the composition for oral consumption consists essentially of (i) (a) methylated-β-cyclodextrin, (b) cannabidiol, and (c) water, (ii) (a) hydroxypropylated-β-cyclodextrin, (b) cannabidiol, and (c) water, or (iii) (a) methylated-β-cyclodextrin, (b) hydroxypropylated-β-cyclodextrin, (c) cannabidiol, and (d) water, other components that exert a material effect (e.g., modify the solubility of cannabidiol) are excluded from the composition for oral consumption, with the exception of trace amounts of ethanol. When the composition for oral consumption consists of (i) (a) methylated-β-cyclodextrin, (b) cannabidiol, and (c) water, (ii) (a) hydroxypropylated-β-cyclodextrin, (b) cannabidiol, and (c) water, or (iii) (a) methylated-β-cyclodextrin, (b) hydroxypropylated-β-cyclodextrin, (c) cannabidiol, and (d) water, the composition excludes any other components, including ethanol.

In some embodiments, the invention provides an alcoholic beverage for oral consumption. The alcoholic beverage for oral consumption comprises, consists essentially of, or consists of (a) methylated-β-cyclodextrin and/or hydroxypropylated-β-cyclodextrin, (b) cannabidiol, (c) ethanol, and (d) water. The alcoholic beverage for oral consumption can comprise ethanol in an amount from about 5 mL to about 50 mL per 100 mL of water. For example, the mixture can comprise ethanol in an amount from about 5 mL to about 40 mL per 100 mL of water, from about 5 mL to about 30 mL per 100 mL of water, from about 5 mL to about 20 mL per 100 mL of water, from about 10 mL to about 50 mL per 100 mL of water, from about 10 mL to about 40 mL per 100 mL of water, from about 10 mL to about 30 mL per 100 mL of water, or from about 10 mL to about 20 mL per 100 mL of water. The relative amounts of methylated-β-cyclodextrin, hydroxypropylated-β-cyclodextrin, and cannabidiol present in the alcoholic beverage for oral consumption are consistent with the composition for oral consumption, described herein.

The alcoholic beverage for oral consumption can further comprise one or more pharmaceutically acceptable excipients. Suitable excipients and the amounts to use may be readily determined under the direction of one of ordinary skill in the art based upon experience and consideration of standard procedures and reference works in the field, e.g., sweetening agents, dyes, flavoring agents, and preservatives.

When the alcoholic beverage for oral consumption consists essentially of (a) methylated-β-cyclodextrin and/or hydroxypropylated-β-cyclodextrin, (b) cannabidiol, (c) ethanol, and (d) water, other components that exert a material effect (e.g., modify the solubility of cannabidiol) are excluded from the composition for oral consumption. When the composition for oral consumption consists of (a) methylated-β-cyclodextrin and/or hydroxypropylated-β-cyclodextrin, (b) cannabidiol, (c) ethanol, and (d) water, the composition excludes any other components.

The invention further provides a method of making a composition for oral consumption. In preferred embodiments, the method comprises (i) adding methylated-β-cyclodextrin and/or hydroxypropylated-β-cyclodextrin to water to form a solution of methylated-β-cyclodextrin and/or hydroxypropylated-β-cyclodextrin in water; (ii) adding cannabidiol to ethanol to form a solution of cannabidiol in ethanol; (iii) adding the solution of cannabidiol in ethanol to the solution of methylated-β-cyclodextrin and/or hydroxypropylated-β-cyclodextrin in water (e.g., over a period of at least about 15 minutes) to form a mixture; (iv) stirring the mixture for a period of, e.g., about 6 hours to about 24 hours; (v) filtering the mixture; and (vi) removing the ethanol from the mixture to form the composition for oral consumption comprising (a) methylated-β-cyclodextrin and/or hydroxypropylated-β-cyclodextrin, (b) cannabidiol, and (c) water.

The method comprises adding methylated-β-cyclodextrin and/or hydroxypropylated-β-cyclodextrin to water to form a solution of methylated-β-cyclodextrin and/or hydroxypropylated-β-cyclodextrin in water. The methylated-β-cyclodextrin and/or hydroxypropylated-β-cyclodextrin can be added to water by any suitable method. For example, the methylated-β-cyclodextrin and/or hydroxypropylated-β-cyclodextrin can be added portion wise or all at once using any suitable apparatus. The solution of methylated-β-cyclodextrin and/or hydroxypropylated-β-cyclodextrin in water can comprise any concentration of methylated-β-cyclodextrin and/or hydroxypropylated-β-cyclodextrin such that the ratio of cannabidiol to methylated-β-cyclodextrin and/or hydroxypropylated-β-cyclodextrin and the final concentration of cannabidiol in the composition for oral consumption are consistent with the values described herein.

The method comprises adding cannabidiol to ethanol to form a solution of cannabidiol in ethanol. The cannabidiol can be added to ethanol by any suitable method. For example, the cannabidiol can be added portion wise or all at once using any suitable apparatus. The solution of cannabidiol in ethanol can comprise any concentration of cannabidiol such that the ratio of cannabidiol to methylated-β-cyclodextrin and/or hydroxypropylated-β-cyclodextrin and the final concentration of cannabidiol in the composition for oral consumption are consistent with the values described herein.

Steps (i) and (ii) of the method can occur in any chronological order. For example, the solution of methylated-β-cyclodextrin and/or hydroxypropylated-β-cyclodextrin in water can be prepared before, after, or at the same time as the solution of cannabidiol in ethanol.

The method comprises adding the solution of cannabidiol in ethanol to the solution of methylated-β-cyclodextrin and/or hydroxypropylated-β-cyclodextrin in water over a period of at least about 15 minutes to form a mixture. The solution of cannabidiol in ethanol can be added to the solution of methylated-β-cyclodextrin and/or hydroxypropylated-β-cyclodextrin in water using any suitable apparatus, such that the solution of cannabidiol in ethanol is preferably added over a period of at least about 15 minutes (e.g., at least about 30 minutes, at least about 1 hour, at least about 2 hours, at least about 3 hours, at least about 6 hours, at least about 12 hours, or at least about 24 hours). In some embodiments, the cannabidiol in ethanol is added to the solution of methylated-β-cyclodextrin and/or hydroxypropylated-β-cyclodextrin in water over a period of at least 30 minutes. Without wishing to be bound by any particular theory, it is believed that if the solution of cannabidiol in ethanol is added too quickly to the solution of methylated-β-cyclodextrin and/or hydroxypropylated-β-cyclodextrin in water, the resulting mixture will produce an insoluble precipitate. The upper limit of the period of time it takes to add the solution of cannabidiol in ethanol to the solution of methylated-β-cyclodextrin and/or hydroxypropylated-β-cyclodextrin in water is not particularly limited. For example, the solution of cannabidiol in ethanol can be added to the solution of methylated-β-cyclodextrin and/or hydroxypropylated-β-cyclodextrin in water over a period of about 1 week or less (e.g., about 5 days or less, about 1 day or less, about 12 hours or less, or about 6 hours or less).

The ratio of ethanol to water in the mixture is not particularly limited, such that the mixture can have any suitable ratio of ethanol to water. However, limiting the amount of ethanol in the mixture can help simplify the step of removing the ethanol from the mixture. Typically, the ratio of ethanol to water in the mixture is from about 1:20 volume to volume (“v/v”) to about 1:2 v/v (e.g., from about 1:20 v/v to about 1:4 v/v, from about 1:20 v/v to about 1:5 v/v, from about 1:10 v/v to about 1:2 v/v, from about 1:10 v/v to about 1:4 v/v, or from about 1:10 v/v to about 1:5 v/v).

A benefit of the method described herein is that the mixture does not need to be stirred more than about 24 hours to form the complex of cannabidiol and methylated-β-cyclodextrin and/or hydroxypropylated-β-cyclodextrin. Thus, the method comprises stirring the mixture for a period of, e.g., from about 6 hours to about 24 hours (e.g., about 8 hours to about 24 hours, about 12 hours to about 24 hours, about 16 hours to about 24 hours, about 20 hours to about 24 hours, about 6 hours to about 18 hours, or about 6 hours to about 12 hours).

The method comprises filtering the mixture. The mixture can be filtered by any suitable technique to remove residual particulates in the mixture. For example, the mixture can be filtered using a filter (e.g., a microfiber filter) with a particle retention of at least about 0.1 μm (e.g., at least about 0.2 μm, at least about 0.3 μm, at least about 0.4 μm, at least about 0.5 μm, at least about 0.6 μm, at least about 0.7 μm, at least about 0.8 μm, at least about 0.9 μm, or at least about 1μm). In preferred embodiments, the mixture is filter using a microfiber filter with a particle retention of at least about 0.7 μm.

The method optionally comprises removing the ethanol from the mixture to form a non-alcoholic product. The ethanol can be removed by any suitable method. For example, the ethanol can be removed under reduced pressure. Any suitable amount of ethanol can be removed such that the composition for oral consumption comprises about 5 wt. % or less (e.g., about 4 wt. % or less, about 3 wt. % or less, about 2 wt. % or less, about 1 wt. % or less, about 0.1 wt. % or less, about 0.01 wt. % or less, or about 0.001 wt. % or less). In preferred embodiments, the method comprises removing all of the ethanol from the mixture, such that the composition for oral consumption is substantially free of ethanol. However, if desired, ethanol can be retained to provide an alcoholic beverage for oral consumption, described herein.

In some embodiments, the method further comprises (vii) removing the water from the composition for oral consumption to form a powder. The resulting powder can be packaged and then solubilized in water (e.g., by a user) to form the composition for oral consumption, or the powder can be solubilized in water and ethanol (e.g., by a user) to form the alcoholic beverage for oral consumption.

In some embodiments, the method comprises adding methylated-β-cyclodextrin and not hydroxypropylated-β-cyclodextrin to water to form a solution of methylated-β-cyclodextrin in water. In some embodiments, the method comprises adding hydroxypropylated-β-cyclodextrin and not methylated-β-cyclodextrin to water to form a solution of hydroxypropylated-β-cyclodextrin in water. In certain embodiments, the method comprises adding methylated-β-cyclodextrin and hydroxypropylated-β-cyclodextrin to water to form a solution of methylated-β-cyclodextrin and hydroxypropylated-β-cyclodextrin in water

The amounts of cannabidiol, methylated-β-cyclodextrin, hydroxypropylated-β-cyclodextrin, and water suitable for the methods described here will be readily apparent from the disclosure as a whole.

The invention is further illustrated by the following embodiments.

(1) A composition for oral consumption comprising: (a) methylated-β-cyclodextrin, (b) cannabidiol, and (c) water, wherein the cannabidiol is present in an amount from about 20 mg to about 40 mg per gram of methylated-β-cyclodextrin, and the cannabidiol is present in the composition in an amount from about 0.8 mg/mL to about 1.6 mg/mL.

(2) The composition of embodiment (1), wherein the cannabidiol is present in an amount from about 22 mg to about 38 mg per gram of methylated-β-cyclodextrin.

(3) The composition of embodiment (2), wherein the cannabidiol is present in an amount from about 25 mg to about 35 mg per gram of methylated-β-cyclodextrin.

(4) The composition of embodiment (3), wherein the cannabidiol is present in an amount from about 25 mg to about 32 mg per gram of methylated-β-cyclodextrin.

(5) The composition of embodiment (4), wherein the cannabidiol is present in an amount from about 25 mg to about 30 mg per gram of methylated-β-cyclodextrin.

(6) The composition of any one of embodiments (1)-(5), wherein the cannabidiol is present in the composition in an amount from about 0.9 mg/mL to about 1.5 mg/mL.

(7) The composition of embodiment (6), wherein the cannabidiol is present in the composition in an amount from about 1 mg/mL to about 1.4 mg/mL.

(8) The composition of embodiment (7), wherein the cannabidiol is present in the composition in an amount from about 1.1 mg/mL to about 1.3 mg/mL.

(9) The composition of embodiment (8), wherein the cannabidiol is present in the composition in an amount of about 1.2 mg/mL.

(10) The composition of any one of embodiments (1)-(9), wherein the composition further comprises one or more pharmaceutically acceptable excipients.

(11) A composition for oral consumption comprising: (a) hydroxypropylated-β-cyclodextrin, (b) cannabidiol, and (c) water, wherein the cannabidiol is present in an amount from about 0.1 mg to about 3.5 mg per gram of hydroxypropylated-β-cyclodextrin, and the cannabidiol is present in the composition in an amount from about 0.01 mg/mL to about 0.13 mg/mL.

(12) The composition of embodiment (11), wherein the cannabidiol is present in an amount from about 0.1 mg to about 3 mg per gram of hydroxypropylated-β-cyclodextrin.

(13) The composition of embodiment (12), wherein the cannabidiol is present in an amount from about 0.1 mg to about 2.5 mg per gram of hydroxypropylated-β-cyclodextrin.

(14) The composition of embodiment (13), wherein the cannabidiol is present in an amount from about 0.1 mg to about 2 mg per gram of hydroxypropylated-β-cyclodextrin.

(15) The composition of embodiment (14), wherein the cannabidiol is present in an amount from about 0.1 mg to about 1.5 mg per gram of hydroxypropylated-β-cyclodextrin.

(16) The composition of any one of embodiments (11)-(15), wherein the cannabidiol is present in the composition in an amount from about 0.05 mg/mL to about 0.13 mg/mL.

(17) The composition of embodiment (16), wherein the cannabidiol is present in the composition in an amount from about 0.1 mg/mL to about 0.13 mg/mL.

(18) The composition of embodiment (17), wherein the cannabidiol is present in the composition in an amount from about 0.11 mg/mL to about 0.13 mg/mL.

(19) The composition of embodiment (18), wherein the cannabidiol is present in the composition in an amount of about 0.13 mg/mL.

(20) The composition of any one of embodiments (11)-(19), wherein the composition further comprises one or more pharmaceutically acceptable excipients.

(21) A method of making a composition for oral consumption comprising: (i) adding methylated-β-cyclodextrin and/or hydroxypropylated-β-cyclodextrin to water to form a solution of methylated-β-cyclodextrin and/or hydroxypropylated-β-cyclodextrin in water; (ii) adding cannabidiol to ethanol to form a solution of cannabidiol in ethanol; (iii) adding the solution of cannabidiol in ethanol to the solution of methylated-β-cyclodextrin and/or hydroxypropylated-β-cyclodextrin in water over a period of at least about 15 minutes to form a mixture; (iv) stirring the mixture for a period of about 6 hours to about 24 hours; (v) filtering the mixture; and (vi) removing the ethanol from the mixture to form the composition for oral consumption comprising (a) methylated-β-cyclodextrin and/or hydroxypropylated-β-cyclodextrin, (b) cannabidiol, and (c) water.

(22) The method of embodiment (21), wherein the composition comprises methylated-β-cyclodextrin and not hydroxypropylated-β-cyclodextrin.

(23) The method of embodiment (22), wherein the cannabidiol is present in an amount from about 20 mg to about 40 mg per gram of methylated-β-cyclodextrin.

(24) The method of embodiment (23), wherein the cannabidiol is present in an amount from about 25 mg to about 35 mg per gram of methylated-β-cyclodextrin.

(25) The method of any one of embodiments (22)-(24), wherein the cannabidiol is present in the composition in an amount from about 0.8 mg/mL to about 1.6 mg/mL.

(26) The method of embodiment (25), wherein the cannabidiol is present in the composition in an amount from about 1 mg/mL to about 1.4 mg/mL.

(27) The method of embodiment (26), wherein the cannabidiol is present in the composition in an amount of about 1.2 mg/mL.

(28) The method of embodiment (21), wherein the composition comprises hydroxypropylated-β-cyclodextrin and not methylated-β-cyclodextrin.

(29) The method of embodiment (28), wherein the cannabidiol is present in an amount from about 0.1 mg to about 3.5 mg per gram of hydroxypropylated-β-cyclodextrin.

(30) The method of embodiment (29), wherein the cannabidiol is present in an amount from about 0.1 mg to about 3 mg per gram of hydroxypropylated-β-cyclodextrin.

(31) The method of any one of embodiments (28)-(30), wherein the cannabidiol is present in the composition in an amount from about 0.01 mg/mL to about 0.13 mg/mL.

(32) The method of embodiment (31), wherein the cannabidiol is present in the composition in an amount from about 0.1 mg/mL to about 0.13 mg/mL.

(33) The method of embodiment (32), wherein the cannabidiol is present in the composition in an amount of about 0.13 mg/mL.

(34) The method of any one of embodiments (21)-(33), wherein the cannabidiol in ethanol is added to the solution of methylated-β-cyclodextrin and/or hydroxypropylated-β-cyclodextrin in water over a period of at least 30 minutes.

(35) The composition of embodiment (1), wherein the composition is formed from a mixture comprising: (a) methylated-β-cyclodextrin, (b) cannabidiol, (c) ethanol, and (d) water, wherein the mixture comprises cannabidiol in an amount from about 20 mg to about 40 mg per gram of methylated-β-cyclodextrin, and ethanol in an amount from about 5 mL to about 50 mL per 100 mL of water.

(36) The composition of embodiment (2), wherein the composition is formed from a mixture comprising: (a) methylated-β-cyclodextrin, (b) cannabidiol, (c) ethanol, and (d) water, wherein the mixture comprises cannabidiol in an amount from about 22 mg to about 38 mg per gram of methylated-β-cyclodextrin, and ethanol in an amount from about 5 mL to about 50 mL per 100 mL of water.

(37) The composition of embodiment (3), wherein the composition is formed from a mixture comprising: (a) methylated-β-cyclodextrin, (b) cannabidiol, (c) ethanol, and (d) water, wherein the mixture comprises cannabidiol in an amount from about 25 mg to about 35 mg per gram of methylated-β-cyclodextrin, and ethanol in an amount from about 5 mL to about 50 mL per 100 mL of water.

(38) The composition of embodiment (4), wherein the composition is formed from a mixture comprising: (a) methylated-β-cyclodextrin, (b) cannabidiol, (c) ethanol, and (d) water, wherein the mixture comprises cannabidiol in an amount from about 25 mg to about 32 mg per gram of methylated-β-cyclodextrin, and ethanol in an amount from about 5 mL to about 50 mL per 100 mL of water.

(39) The composition of embodiment (5), wherein the composition is formed from a mixture comprising: (a) methylated-β-cyclodextrin, (b) cannabidiol, (c) ethanol, and (d) water, wherein the mixture comprises cannabidiol in an amount from about 25 mg to about 30 mg per gram of methylated-β-cyclodextrin, and ethanol in an amount from about 5 mL to about 50 mL per 100 mL of water.

(40) The composition of any one of embodiments (35)-(39), the mixture comprises ethanol in an amount from about 10 mL to about 20 mL per 100 mL of water.

(41) The composition of embodiment (11), wherein the composition is formed from a mixture comprising: (a) hydroxypropylated-β-cyclodextrin, (b) cannabidiol, (c) ethanol, and (d) water, wherein the mixture comprises cannabidiol in an amount from about 0.1 mg to about 3.5 mg per gram of hydroxypropylated-β-cyclodextrin, and ethanol in an amount from about 5 mL to about 50 mL per 100 mL of water.

(42) The composition of embodiment (12), wherein the composition is formed from a mixture comprising: (a) hydroxypropylated-β-cyclodextrin, (b) cannabidiol, (c) ethanol, and (d) water, wherein the mixture comprises cannabidiol in an amount from about 0.1 mg to about 3 mg per gram of hydroxypropylated-β-cyclodextrin, and ethanol in an amount from about 5 mL to about 50 mL per 100 mL of water.

(43) The composition of embodiment (13), wherein the composition is formed from a mixture comprising: (a) hydroxypropylated-β-cyclodextrin, (b) cannabidiol, (c) ethanol, and (d) water, wherein the mixture comprises cannabidiol in an amount from about 0.1 mg to about 2.5 mg per gram of hydroxypropylated-β-cyclodextrin, and ethanol in an amount from about 5 mL to about 50 mL per 100 mL of water.

(44) The composition of embodiment (14), wherein the composition is formed from a mixture comprising: (a) hydroxypropylated-β-cyclodextrin, (b) cannabidiol, (c) ethanol, and (d) water, wherein the mixture comprises cannabidiol in an amount from about 0.1 mg to about 2 mg per gram of hydroxypropylated-β-cyclodextrin, and ethanol in an amount from about 5 mL to about 50 mL per 100 mL of water.

(45) The composition of embodiment (15), wherein the composition is formed from a mixture comprising: (a) hydroxypropylated-β-cyclodextrin, (b) cannabidiol, (c) ethanol, and (d) water, wherein the mixture comprises cannabidiol in an amount from about 0.1 mg to about 1.5 mg per gram of hydroxypropylated-β-cyclodextrin, and ethanol in an amount from about 5 mL to about 50 mL per 100 mL of water.

(46) The composition of any one of embodiments (41)-(45), the mixture comprises ethanol in an amount from about 10 mL to about 20 mL per 100 mL of water.

The following examples further illustrate the invention but, of course, should not be construed as in any way limiting its scope.

### EXAMPLE 1

This example illustrates concentration of methyl-β-cyclodextrin and cannabidiol (“CBD”) in the preparation of a formulation for oral consumption and a powder for oral consumption.

Methyl-β-cyclodextrin (5 g) was added to 100 ml of reverse osmosis purified (“R.O.”) water in a 250 ml beaker and stirred to dissolve. While stirring, solid cannabidiol (68 mg, 150 mg, or 240 mg), dissolved in 15 ml of food grade ethanol, was added dropwise using an addition funnel to avoid precipitation. For the complete addition of the cannabidiol solution, 30 minutes were required.

The reaction mixture was stirred at room temperature overnight (24 hours). The resulting reaction mixture was turbid. The reaction mixture was filtered with a Buchner funnel using Whatman® Glass Microfiber Filter GF/F (Diameter 110 mm, particle retention 0.7 μm) to afford a clear solution.

The resulting filtrate (120 mL) was analyzed by HPLC and the results are summarized in Table 1 and plotted in the FIGURE.

TABLE 1

| Amount of | Amount | Total volume | Concentration |
|---|---|---|---|
| methyl-β- | of CBD | of methyl-β- | of methyl-β- |
| cyclodextrin | in 15 mL | cyclodextrin/ | cyclodextrin/ |
| in 100 mL | of ethanol | CBD | CBD |
| of water (g) | (mg) | mixture (ml) | (mg/ml) |
| 5 | 68 | 120 | 0.55 |
| 5 | 150 | 120 | 1.2 |
| 5 | 240 | 120 | 1.32 |

The FIGURE shows that the concentration of the methyl-β-cyclodextrin/CBD complex begins to plateau at a concentration of 1.2 mg/mL, which corresponds to a ratio of cannabidiol to methyl-β-cyclodextrin of about 30 mg/g. Accordingly, the FIGURE demonstrates that in order to achieve a high concentration of methyl-β-cyclodextrin/CBD complex in solution using a relatively low amount of methyl-β-cyclodextrin and cannabidiol, the ratio of cannabidiol to methyl-β-cyclodextrin can be about 30 mg/g.

The ethanol can be removed under reduced pressure to afford a composition for oral consumption comprising methyl-β-cyclodextrin, cannabidiol, and water. Alternatively, or in addition to, the water can be removed under reduced pressure to afford a powder for oral consumption comprising methyl-β-cyclodextrin and cannabidiol.

### EXAMPLE 2

This example illustrates concentration of 2-hydroxypropyl-β-cyclodextrin and cannabidiol (“CBD”) in the preparation of a composition for oral consumption and a powder for oral consumption.

2-hydroxypropyl-β-cyclodextrin (20 g or 37.5 g) was added to reverse osmosis purified (“R.O.”) water (100 mL or 750 mL) in a beaker and stirred to dissolve. While stirring, solid cannabidiol (57 mg, 150 mg, 380 mg, or 1.5 g), dissolved in food grade ethanol (15 mL or 100 mL), was added dropwise using an addition funnel to avoid precipitation. For the complete addition of the cannabidiol solution, 30 minutes were required.

The reaction mixture was stirred at room temperature overnight (24 hours). The resulting reaction mixture was turbid. The reaction mixture was filtered with a Buchner funnel using Whatman® Glass Microfiber Filter GF/F (Diameter 110 mm, particle retention 0.7 μm).

The resulting filtrate (130 mL or 850) was analyzed by HPLC and the results are summarized in Table 2.

TABLE 2

| | | Total volume | Concentration |
|---|---|---|---|
| | | of 2- | of 2- |
| | | hydroxypropyl- | hydroxypropyl- |
| Amount of 2- | | β-cyclodextrin/ | β-cyclodextrin/ |
| hydroxypropyl- | Amount | CBD | CBD |
| β-cyclodextrin | of CBD | mixture (ml) | (mg/ml) |
| 37.5 g in 750 | 1.5 g in 100 ml | 850 | 0.13 |
| mL of water | of ethanol | | |
| 37.5 g in 750 | 380 mg in 100 | 850 | 0.13 |
| mL of water | ml of ethanol | | |
| 20 g in 100 ml | 150 mg in 15 ml | 130 | 0.13 |
| of water | of ethanol) | | |
| 20 g in 100 ml | 57 mg in 15 ml | 130 | 0.13 |
| of water | of ethanol | | |

Table 2 shows that the concentration of the 2-hydroxypropyl-β-cyclodextrin/CBD complex is 0.13 mg/mL for all ratios of cannabidiol to 2-hydroxypropyl-β-cyclodextrin measured. Accordingly, Table 2 demonstrates that in order to achieve a high concentration of 2-hydroxypropyl-β-cyclodextrin/CBD complex in solution using a relatively low amount of 2-hydroxypropyl-β-cyclodextrin and cannabidiol, the ratio of cannabidiol to 2-hydroxypropyl-β-cyclodextrin can be less than about 3 mg/g.

The ethanol can be removed under reduced pressure to afford a composition for oral consumption comprising 2-hydroxypropyl-β-cyclodextrin, cannabidiol, and water. Alternatively, or in addition to, the water can be removed under reduced pressure to afford a powder for oral consumption comprising 2-hydroxypropyl-β-cyclodextrin and cannabidiol.

### EXAMPLE 3

This example provides an exemplary preparation of a composition for oral consumption from a powder for oral consumption. The resulting composition for oral consumption contains 10 mg of cannabidiol (“CBD”) in 500 mL of water 3.3 g of water-soluble CBD powder (contains 0.3% CBD) was added to Member's Mark® Purified Water (500 mL) and mixed. The resulting composition for oral consumption comprising 2-hydroxypropyl-β-cyclodextrin, cannabidiol, and water was clear. The composition for oral consumption was analyzed by HPLC. The concentration of CBD was 0.02 mg/mL and the concentration of 2-hydroxypropyl β-cyclodextrin was 6.6 mg/mL.

### EXAMPLE 4

This example provides an exemplary preparation of a composition for oral consumption from a powder for oral consumption. The resulting composition for oral consumption contains 50 mg of cannabidiol (“CBD”) in 500 mL of water 16.67 g of water-soluble CBD powder (contains 0.3% CBD) was added to Member's Mark® Purified Water (500 mL) and mixed. The resulting composition for oral consumption comprising 2-hydroxypropyl-β-cyclodextrin, cannabidiol, and water was clear. The composition for oral consumption was analyzed by HPLC. The concentration of CBD was 0.1 mg/mL and the concentration of 2-hydroxypropyl β-cyclodextrin was 33.3 mg/mL.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition for oral consumption comprising:
(a) methylated-β-cyclodextrin,
(b) cannabidiol, and
(c) water,
wherein the cannabidiol is present in an amount from about 20 mg to about 40 mg per gram of methylated-β-cyclodextrin, and the cannabidiol is present in the composition in an amount from about 0.8 mg/mL to about 1.6 mg/mL.

2. The composition of claim 1, wherein the cannabidiol is present in an amount from about 25 mg to about 35 mg per gram of methylated-β-cyclodextrin.

3. The composition of claim 2, wherein the cannabidiol is present in an amount from about 25 mg to about 30 mg per gram of methylated-β-cyclodextrin.

4. The composition of claim 1, wherein the cannabidiol is present in the composition in an amount from about 1 mg/mL to about 1.4 mg/mL.

5. The composition of claim 4, wherein the cannabidiol is present in the composition in an amount of about 1.2 mg/mL.

6. The composition of claim 1, wherein the composition further comprises one or more pharmaceutically acceptable excipients.

7. The composition of claim 1, wherein the composition is formed from a mixture comprising:
(a) methylated-β-cyclodextrin,
(b) cannabidiol,
(c) ethanol, and
(d) water,
wherein the mixture comprises cannabidiol in an amount from about 20 mg to about 40 mg per gram of methylated-β-cyclodextrin, and ethanol in an amount from about 5 mL to about 50 mL per 100 mL of water.

8. The composition of claim 7, wherein the mixture comprises ethanol in an amount from about 10 mL to about 20 mL per 100 mL of water.

9. The composition of claim 3, wherein the composition is formed from a mixture comprising:
(a) methylated-β-cyclodextrin,
(b) cannabidiol,
(c) ethanol, and
(d) water,
wherein the mixture comprises cannabidiol in an amount from about 25 mg to about 30 mg per gram of methylated-β-cyclodextrin, and ethanol in an amount from about 5 mL to about 50 mL per 100 mL of water.

10. The composition of claim 9, wherein the mixture comprises ethanol in an amount from about 10 mL to about 20 mL per 100 mL of water.

11. A composition for oral consumption comprising:
(a) hydroxypropylated-β-cyclodextrin,
(b) cannabidiol, and
(c) water,
wherein the cannabidiol is present in an amount from about 0.1 mg to about 3.5 mg per gram of hydroxypropylated-β-cyclodextrin, and the cannabidiol is present in the composition in an amount from about 0.01 mg/mL to about 0.13 mg/mL.

12. The composition of claim 11, wherein the cannabidiol is present in an amount from about 0.1 mg to about 2.5 mg per gram of hydroxypropylated-β-cyclodextrin.

13. The composition of claim 12, wherein the cannabidiol is present in an amount from about 0.1 mg to about 1.5 mg per gram of hydroxypropylated-β-cyclodextrin.

14. The composition of claim 11, wherein the cannabidiol is present in the composition in an amount from about 0.05 mg/mL to about 0.13 mg/mL.

15. The composition of claim 14, wherein the cannabidiol is present in the composition in an amount of about 0.13 mg/mL.

16. The composition of claim 11, wherein the composition further comprises one or more pharmaceutically acceptable excipients.

17. The composition of claim 11, wherein the composition is formed from a mixture comprising:
(a) hydroxypropylated-β-cyclodextrin,
(b) cannabidiol,
(c) ethanol, and
(d) water,
wherein the mixture comprises cannabidiol in an amount from about 0.1 mg to about 3.5 mg per gram of hydroxypropylated-β-cyclodextrin, and ethanol in an amount from about 5 mL to about 50 mL per 100 mL of water.

18. The composition of claim 17, wherein the mixture comprises ethanol in an amount from about 10 mL to about 20 mL per 100 mL of water.

19. The composition of claim 13, wherein the composition is formed from a mixture comprising:
(a) hydroxypropylated-β-cyclodextrin,
(b) cannabidiol,
(c) ethanol, and
(d) water,
wherein the mixture comprises cannabidiol in an from about 0.1 mg to about 1.5 mg per gram of hydroxypropylated-β-cyclodextrin, and ethanol in an amount from about 5 mL to about 50 mL per 100 mL of water.

20. The composition of claim 19, wherein the mixture comprises ethanol in an amount from about 10 mL to about 20 mL per 100 mL of water.

21. A method of making a composition for oral consumption comprising:
(i) adding methylated-β-cyclodextrin and/or hydroxypropylated-β-cyclodextrin to water to form a solution of methylated-β-cyclodextrin and/or hydroxypropylated-β-cyclodextrin in water;
(ii) adding cannabidiol to ethanol to form a solution of cannabidiol in ethanol;
(iii) adding the solution of cannabidiol in ethanol to the solution of methylated-β-cyclodextrin and/or hydroxypropylated-β-cyclodextrin in water over a period of at least about 15 minutes to form a mixture;
(iv) stirring the mixture for a period of about 6 hours to about 24 hours;
(v) filtering the mixture; and
(vi) removing the ethanol from the mixture to form the composition for oral consumption comprising (a) methylated-β-cyclodextrin and/or hydroxypropylated-β-cyclodextrin, (b) cannabidiol, and (c) water.

22. The method of claim 21, wherein the composition comprises methylated-β-cyclodextrin and not hydroxypropylated-β-cyclodextrin.

23. The method of claim 22, wherein the cannabidiol is present in an amount from about 20 mg to about 40 mg per gram of methylated-β-cyclodextrin.

24. The method of claim 23, wherein the cannabidiol is present in the composition in an amount from about 0.8 mg/mL to about 1.6 mg/mL.

25. The method of claim 24, wherein the cannabidiol is present in the composition in an amount of about 1.2 mg/mL.

26. The method of claim 21, wherein the composition comprises hydroxypropylated-β-cyclodextrin and not methylated-β-cyclodextrin.

27. The method of claim 26, wherein the cannabidiol is present in an amount from about 0.1 mg to about 3.5 mg per gram of hydroxypropylated-β-cyclodextrin.

28. The method of claim 27, wherein the cannabidiol is present in the composition in an amount from about 0.01 mg/mL to about 0.13 mg/mL.

29. The method of claim 28, wherein the cannabidiol is present in the composition in an amount of about 0.13 mg/mL.

30. The method of claim 21, wherein the cannabidiol in ethanol is added to the solution of methylated-β-cyclodextrin and/or hydroxypropylated-β-cyclodextrin in water over a period of at least 30 minutes.
